(19) United States
(12) Patent Application Publication
(10) Pub. No.: US 2015/0258141 A1
(43) Pub. Date: Sep. 17, 2015

(54) ELECTROLYTIC SPORTS DRINK COMPOSITION

(71) Applicant: Carl J. Randolph, Stafford, TX (US)

(72) Inventor: Carl J. Randolph, Stafford, TX (US)

(21) Appl. No.: 14/660,754

(22) Filed: Mar. 17, 2015

Related U.S. Application Data
(60) Provisional application No. 61/954,468, filed on Mar. 17, 2014.

(51) Int. Cl. A61K 33/24; A61K 33/00; A61K 33/06; A23L 2/52; A61K 31/375; A61K 31/355; A61K 31/7016; A61K 9/00; A61K 31/714

(52) U.S. Cl. CPC A61K 33/24; A61K 9/0095; A61K 33/00; A61K 33/06; A61K 31/714; A61K 31/375; A61K 31/355; A61K 31/7016; A61K 9/0053; A23L 2/52; A23V 2002/00

(57) ABSTRACT

A composition taken before, during, or after exercise; comprising: potassium; sodium; magnesium; chromium; vitamin B12; vitamin C; vitamin E; and sucrose. A method of formulating a composition including potassium; sodium; magnesium; chromium; vitamin B12; vitamin C; vitamin E; and sucrose. The composition is believed to be significant to athletes with sickle cell disease (SCD) and/or sickle cell trait to avoid dehydration and aid in muscle recovery.

### CROSS REFERENCE TO RELATED APPLICATION

This application claims the benefit of U.S. Provisional Application No. 61/954,468 filed Mar. 17, 2014, herein incorporated by reference in its entirety for all purposes.

### FIELD OF THE INVENTION

The present invention relates to compositions for maximizing hydration during athletic activity while also extending endurance and aiding in maximizing muscle performance and recovery.

### BACKGROUND OF THE INVENTION

Exercise acts to deplete the body of fuel stores and increases the rate of perspiration causing loss of water and mineral salts. These losses are known to have physical effects on the athlete if exercise is prolonged and particularly if ambient temperatures are moderate or high. Dehydration, fatigue, and cramping may act to limit or restrict performance.

The efficacy and timing of replacement of liquids and metabolites during and immediately after exercise is of importance. Repletion of some or all of the lost energy, water and salts will not only restore the body to its normal balance but can sustain, enhance and restore performance. This is particularly relevant for competitive sports or activities such as football wherein fatigue is a primary limiting factor for overall performance.

Sustained hydration or rehydration can be accomplished in a number of different ways. In the most basic sense, water replaces some of the fluids lost through sweat and helps maintain body temperature and important cardiovascular functions. There are currently available a number of beverages, and concentrated beverage compositions (including liquid, powdered or capsule concentrates) to be prepared by the consumer into a drinkable form, which are used for rehydration of fluids lost during exercise. These rehydration beverages (also known as “sports drinks”), both in the premixed (or ready-to-drink) form and as prepared by the user, may be consumed before, during and after exercise.

Sports drinks replace the fluids and electrolytes lost through sweat. This is an improvement over plain water not only because these drinks replace some of the minerals lost in sweat but also because carbohydrates are provided as a source of added energy. Sports drinks containing inorganic salts to replace those lost as sweat are well established. Drinks having a high concentration of sugar are also common but a high sugar content is undesirable and may produce a solution having a high osmolality, preventing the fluid from rapidly entering a person's body from the stomach. In fact, fluid may be drawn in the opposite direction. Sports drinks containing a high concentration of sugar are not effective. While conventional sports drinks rehydrate the body better than plain water, there remains a need for a superior hydration or rehydration composition that also contains less or no added sugar or like sweetener.

Sickle cell disease (SCD), generally includes a group of conditions which are genetic (inherited). SCD affects the red blood cells in the blood. Sickle cell anemia is the name of a specific form of SCD in which there are two sickle cell genes. Sickle cell disease turns normal, round blood cells into misshaped red blood cells that look like sickles or crescent moons. These sickled cells can get stuck in blood vessels, blocking blood flow and causing severe pain as well as damage to organs, muscles, and bones. Dehydration generally, and particularly during exercise, can trigger blood flow to slow down, which may cause a painful event in a person with sickle cell disease.

Sickle cell anemia is inherited from both parents. If the sickle cell gene is inherited from only one parent, it is termed sickle cell trait. People with sickle cell trait commonly do not have the symptoms of sickle cell anemia.

Sickle cell anemia is caused by an abnormal type of hemoglobin called hemoglobin S. Hemoglobin is a protein inside red blood cells that carries oxygen. Hemoglobin S changes the red blood cells from their normal shape to become fragile and shaped like crescents or sickles. The sickled cells deliver less oxygen to the body's tissues which could trigger a potentially dangerous condition for an athlete. These sickled red blood cells have a life which is much shorter than red blood cells, causing anemia. The sickled red blood cells get stuck in small blood vessels and break into pieces. This can interrupt healthy blood flow and cut down even more on the amount of oxygen flowing to body tissues cause cramping.

SCD may include episodes of symptoms but, in between episodes, the personal generally feels well with no symptoms. The reason for bouts of symptoms is that the red blood cells function normally for much of the time but if some trigger causes too many of them sickle, the sickle cells cause symptoms. The onset of severe and sudden symptoms due to sickling is called a sickle cell crisis.

Almost all people with sickle cell anemia have painful crisis episodes. These crises can last from hours to days causing pain in the lower back, leg, joints, and chest. Some people have one episode every few years. Others have many episodes each year. The crises can be severe enough to require a hospital stay.

Sickling for people with sickle cell anemia is promoted by conditions which are associated with low oxygen levels, increased acidity, or low volume (dehydration) of the blood. These conditions can occur as a result of injury to the body's tissues, dehydration, or anesthesia.

The spleen is an organ located in the abdomen, in the top left-hand side. The function of the spleen is to help the immune system primarily by removing abnormal cells from the bloodstream. Sickle cells can block blood vessels in the spleen. This can make the spleen swell up suddenly with blood, losing blood volume (red blood cells) into the spleen. This is one cause of sudden and severe anemia associated with SCD. The medical term is splenic sequestration requiring urgent medical treatment.

Fear of dehydration should not keep a person with SCD from exercising. However, the athlete must be sure to drink fluids before, during and after physical activities. The athlete and those involved in athletics should be aware of the symptoms of dehydration and what steps should be taken if it occurs. Muscle recovery is also a significant problem. People with SCD often require much greater time for their muscles to recover following exercise. A need therefore exists for a sports drink that assists people with SCD to remain properly hydrated during exercise and assists in muscle recovery following exercise.

Sickle cell trait, which is not considered SCD, is the inheritance of one gene for sickle hemoglobin (hemoglobins) and one for normal hemoglobin. During intense or extensive physical exertion, the sickle hemoglobin can change the shape of red cells from round to quarter-moon. This process is known as “sickling.” This change, exertional sickling, can pose a grave risk for some athletes.

Sickle cell trait is not regarded as a disease state (SCD) because it has complications that are either uncommon or mild. Notwithstanding, under unusual circumstances serious adverse health effects can result from complications related to polymerization of deoxy-hemoglobin S. Such problems include increased urinary tract infection in women, gross hematuria, complications of hyphema, splenic infarction with altitude hypoxia or exercise. In addition, life-threatening complications resulting from exercise exertion, exertional heat illness (exertional rhabdomyolysis, heat stroke, or renal failure) or idiopathic sudden death.

In sickle cell trait, strenuous exercise evokes four forces that in concert foster sickling, 1) severe hypoxemia, 2) metabolic acidosis; 3) hyperthermia in muscles, and 4) red-cell dehydration.

Research shows how and why sickle red cells can accumulate in the bloodstream during intense exercise. Sickle cells can “logjam” blood vessels and lead to collapse from ischemic rhabdomyolysis, the rapid breakdown of muscles starved of blood (oxygen). Major metabolic problems from ischemic rhabdomyolysis can threaten the life of the athlete. Sickling can begin within 2-3 minutes of any all-out exertion—and can reach grave levels soon thereafter if the athlete continues to struggle. Heat, dehydration, altitude, and asthma can increase the risk for and worsen sickling, even when exercise is not considered all-out exertion.

It is believed that the harder and faster athletes with sickle cell trait go, the earlier and greater the sickling. This likely explains why exertional collapse has been found to occur sooner in college football players during or just following sprints than in military recruits who collapsed running longer distances. Sickling can begin after only 2-3 minutes of sprinting, or other such all-out exertional exercise.

Persons with sickle cell trait have been shown to be more vulnerable than those without trait to heat stroke and muscle breakdown (rhabdomyolysis) when subjected to strenuous forced exercise in military training under unfavorable environmental conditions. An important potential complication of sickle cell trait is unexpected exercise-related death (ERD). The possibility that previously healthy young people with sickle cell trait might suffer increased mortality from exercise was first suggested by observations of enlisted recruits in US Armed Forces basic training. A military trainee with Hb AS may suffer exercise related hypernatremia during physical training in the field. This could result in acute renal failure requiring dialysis.

Twelve cases of natural exercise-related death (ERD) among apparently healthy young men with Hb AS were reported by 1981. These deaths were predominantly due to exertional rhabdomyolysis, although some were sudden idiopathic deaths with cardiopulmonary arrest, associated in two cases with hyperkalemia. Identical presentations were observed in recruits without Hb AS. There is no direct proof that sickle cell trait contributed to ERD through microvascular obstruction by rigid erythrocytes. There is little evidence that these deaths involve the typical acute complications of sickle cell disease, such as acute focal infarction of the spleen, kidneys, lungs, bone, retina, or brain, sudden extensive sequestration of blood in the spleen or liver, or overwhelming infection with encapsulated bacteria.

Effective prevention of sickling during demanding physical conditioning requires following measures similar to those used by recruits and distance runners. Performance levels should be built up gradually, avoiding severe muscle pain. Training should cease and restart gradually when substantial myalgia occurs. Adequate hydration with increased fluid intake rising with environmental heat stress is essential. In the evening of any hot day, the athlete should be sure to ingest adequate amounts of at least salt and potassium to replace sweat loss and water to replace fluid deficits. One recommendation includes checking the color of the first AM urine in a clear plastic cup as an easy method to identify people who are dehydrated from prior day heat exposure if measurement of urine specific gravity is not readily available. Those with darker urine can drink an additional pint or quart of water before starting exercise. Athletes in a demanding training program should keep a log of daily weights from the same scale on waking and before going to sleep at night.

Alternatively, over-hydration with water alone is possible with consequent hyponatremia, seizures, and death. Oral hydration should not exceed one quart per hour or 12 quarts per day without monitoring of blood chemistries. Patients with muscle cramps require additional salt and electrolytes, which can be taken orally or intravenously. During sustained exercise, such as marching, middle to long distance running, basketball, and soccer, athletes should drink fluids at intervals of approximately 15-20 minutes.

Sodium and potassium replacement with meals avoids aggravating the common trend toward hypernatremia during exercise in heat. One should be careful to avoid sustained full intensity efforts lasting more than two minutes. Special attention should be given to exercise at full competitive intensity.

A need further exists for a sports drink suitable for people with sickle cell trait to assist in avoiding dehydration during exercise. Additionally, a need exists for a sports drink that aids in muscle recovery for the sickle cell trait athlete.

### SUMMARY OF THE INVENTION

An electrolyte sports drink composition to be taken orally before, during, or after exercise in order to maximize hydration during exercise, extend endurance, and aid in maximizing muscle performance and recovery. The benefits of fully hydrated cells are significant to all athletes. It is believed that in the alternative, the composition of the present disclosure may reduce or eliminate the symptoms of pre-menstrual syndrome (PMS). Further and alternatively, the composition of the present disclosure may assist athletes with SCD or sickle cell trait to remain hydrated during physical exercise and also aid those athletes in muscle recovery.

However, sweat is a mixture of fluids and electrolytes which include sodium. The fluids are secreted when the body needs to cool itself down. Fluid intake should match the sweat rate of the athlete to minimize detrimental effects and performance. Athletes performing high intensity physical activity commonly have sweat rates of 1.0-2.5 L per hour, however, when the ambient temperature is higher, sweat rates many exceed 2.5 L per hour.

Most individuals can tolerate a 2% decrease in body fluid without any significant risk provided they are in a cool or temperate environment, however if this occurs in a hot environment (>30° C.) there can be significant risk to health. The risk is further heightened for the SCD athlete.

When fluid loss occurs from sweating it causes a reduction in blood volume. This thickens the blood, making it harder for the heart to pump the blood around the body. To offset this effect the heart rate increases, but oxygen delivery to exercising muscles and removal of carbon dioxide and waste products from them is hindered. Fluid loss can also lead to an increased core body temperature, which impairs the activity of enzymes in the body.

The natural thirst mechanism includes individuals consume only approximately half the amount of fluid they have lost through sweating. It is thought that the thirst mechanism does not initiate the drive to drink until the body is 2% dehydrated. At this point, athletic performance can be compromised.

A composition taken before, during, or after exercise; including: potassium; sodium; magnesium; chromium; vitamin B12; vitamin C; vitamin E; and sucrose.

A method of preparation of an orally ingestible blend, comprising the steps of:

obtaining in substantially powder form the following constituents: potassium; sodium; magnesium; chromium; vitamin B12; vitamin C; vitamin E; and, sucrose; combining said constituents into a mixture.

The method may also include encapsulated of the composition for oral ingestion.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processes and manufacturing techniques are omitted so as to not unnecessarily obscure the embodiments herein. The examples used herein are intended merely to facilitate an understanding of ways in which the invention herein may be practiced and to further enable those of skill in the art to practice the embodiments herein. Accordingly, the examples should not be construed as limiting the scope of the claimed invention.

Potassium assists in proper heart and muscle function as well as minimizing increases in blood pressure levels resulting from sodium intake. It also assists in proper food digestion. A recommended intake is 4-7 grams per day which may be even higher for the athlete.

Sodium is the major salt component in human sweat. The loss of sodium through sweating could be the major contributor to the development of muscle cramps or weakness for the athlete. Sodium has been determined to stimulate the thirst mechanism. It is also believed to increase the rate at which the small intestine can absorb carbohydrates and water. Once the water is absorbed, sodium is believed to assist in water retention and proper blood fluid chemistry. As a result, sodium assists in rehydration and water retention of the athlete and the delay of muscle fatigue.

It is believed that consuming about 450 mg or more of sodium per hour of exercise is required to maintain blood sodium levels and blood plasma volume.

Magnesium aids in helping muscles relax. Magnesium also aids in glucose metabolism, and it used in many enzymatic reactions. It is believed that magnesium intake for males should be between 330-350 mg per day, and females should intake about 255-265 mg per day. However, when exercising in hot and humid conditions, magnesium stores can be excreted through sweat requiring more to be replenished.

Chromium is an essential mineral that plays a role in how insulin helps the body regulate blood sugar levels. Insulin is a hormone that the body uses to change sugar, starches, and other food into energy needed for sustained athletic performance.

Vitamin B12 is a nutrient that helps keep the body's nerve and blood cells function properly. It also helps make DNA. Vitamin B12 is further believed to help prevent anemia. It is believed that adults should intake at least 2.4 mcg per day.

Vitamin C is the major water-soluble antioxidant within the body. This vitamin is known to readily donate electrons to break the chain reaction of lipid peroxidation. The water-soluble properties of vitamin C allow for the quenching of free radicals before they reach the cellular membrane. The benefits of vitamin C as an antioxidant during exercise is believed to provide prophylactic effects, when free radical formation is high. It is believed that vitamin C further plays a role in exercise recovery. Vitamin C needs are increased in those who exercise. Athletes receiving a vitamin C supplement showed increased work capacity. Vitamin C has shown favorable effects when used during heat acclimatization. Vitamin C supplementation may also be beneficial in helping prevent upper respiratory infections in ultramarathoners. A daily intake between 100-300 mg of vitamin C may be warranted to meet the needs of people who exercise.

Since it is believed that endurance exercise may promote free radical generation in the body, vitamin E may be added in a preferred embodiment. Vitamin E may play an important role in preventing the free radical damage associated with endurance exercise. It is believed that as a result of exercise, vitamin E may prevent oxidative damage. Vitamin E contributes to preventing exercise-induced lipid peroxidation.

Sucrose broken down by the body into glucose may play a significant role in water and sodium absorption for the athlete, particularly when the normal sodium absorption mechanism is impaired or if the athlete is in a diarrheal state. Ingesting a saline solution (water plus sodium) by mouth may have diminished beneficial effect because the normal mechanism by which sodium is absorbed by the healthy intestinal wall is impaired in the diarrheal. In the event the sodium is not absorbed, the water is not absorbed either. If sucrose or glucose is added to a saline solution in a sports drink, it is believed that the sucrose or glucose molecules are absorbed through the intestinal wall, regardless of any diarrheal condition. In addition to the sucrose or glucose, the sodium is carried through by a co-transport coupling mechanism. This occurs in a 1:1 ratio, one molecule of sucrose or glucose co-transporting one sodium ion. The sucrose or glucose does not co-transport water. Instead it is the increased concentration of sodium absorbed across the intestinal wall which pulls water through. In the present disclosure, sucrose may be obtained through the addition of organic cane sugar. In a preferred embodiment, 2.8 grams of organic cane sugar may be added.

An electrolytic formulation of the present invention is preferably prepared in the following compositions:

Composition Materials

| Potassium Range | | |
|---|---|---|
| General | 200-550 | mg |
| More Particular | 350-475 | mg |
| Even More Part. | 325-425 | mg |
| Most Preferred | 400 | mg |
| Sodium Range | | |
| General | 100-350 | mg |
| More Particular | 150-275 | mg |
| Even More Part. | 175-225 | mg |
| Most Preferred | 200 | mg |
| Magnesium Range | | |
| General | 60-200 | mg |
| More Particular | 65-175 | mg |
| Even More Part. | 75-125 | mg |
| Most Preferred | 80 | mg |
| Chromium Range | | |
| General | 1-20 | mcg |
| More Particular | 2-18 | mcg |
| Even More Part. | 5-15 | mcg |
| Most Preferred | .01 mg or 1 | mcg |
| B12 Range | | |
| General | 35-200 | mcg |
| More Particular | 50-150 | mcg |
| Even More Part. | 75-125 | mcg |
| Most Preferred | 1 mg or 100 | mcg |
| Vitamin C Range | | |
| General | 300-700 | mg |
| More Particular | 400-600 | mg |
| Even More Part. | 475-525 | mg |
| Most Preferred | 500 | mg |
| Vitamin E Range | | |
| General | 3-25 | mg |
| More Particular | 5-23 | mg |
| Even More Part. | 8-15 | mg |
| Most Preferred | 10 | mg |
| Sucrose Range | | |
| General | 2-20 | mg |
| More Particular | 5-15 | mg |
| Even More Part. | 8-12 | mg |
| Most Preferred | 10 | mg |

Composition Formula

| | | |
|---|---|---|
| More Particular | | |
| Vitamin C | 600 | mg |
| Potassium | 500 | mg |
| Sodium | 300 | mg |
| Magnesium | 150 | mg |
| Chromium | .05 mg or 5 | mcg |
| B12 | 1.25 mg or 125 | mcg |
| Sucrose | 25 | mg |
| Vitamin E | 25 | mg |
| Even More Particular | | |
| Vitamin C | 550 | mg |
| Potassium | 450 | mg |
| Sodium | 250 | mg |
| Magnesium | 125 | mg |
| Chromium | .07 mg or 7 | mcg |
| B12 | 1.10 mg or 110 | mcg |
| Sucrose | 15 | mg |
| Vitamin E | 15 | mg |
| Most Preferred | | |
| Vitamin C | 500 | mg |
| Potassium | 400 | mg |
| Sodium | 200 | mg |
| Magnesium | 100 | mg |
| Chromium | .01 mg or 1 | mcg |
| B12 | 1 mg or 100 | mcg |
| Sucrose | 10 | mg |
| Vitamin E | 10 | mg |
| Alternate Most Preferred | | |
| Vitamin C | 500 | mg |
| Potassium | 400 | mg |
| Sodium | 200 | mg |
| Magnesium | 80 | mg |
| Chromium | 1 | mcg |
| B12 | 1000 | mcg |

Method of Manufacture of a Composition Product

Powder Form: Each material is bought in bulk. Then exact measurements are taken for each material using a measuring spoon and mixed together. Flavors may also be added. For example, organic beet extract may be added to formulate a fruit punch flavor or monk fruit to formulate a pink lemonade flavor.

Embodiment Formula: Fruit Punch—Pills per serving: 1.

| Ingredient | Amount | | Daily Value |
|---|---|---|---|
| Vitamin C (as Ascorbic Acid) | 500 | mg | 833% |
| Vitamin B12 (as Cyanocobalamin)(1% on DCP) | 1000 | mcg | 16666% |
| Chromium (as Chromium Polynicotinate) | 1 | mcg | 0% |
| Sodium Chloride (40-Sodium/60-Chloride) | 200 | mg | 3% |
| Potassium Chloride (52% Potassium) | 400 | mg | NA |
| Magnesium citrate (trimagnesium dicitrate) | 80 | mg | 80% |
| anhydrous (15% Mg) | | | |

Other Ingredients: Higher Mesh Organic Evaporated Cane Sugar, Carmi Nat. Fruit Punch 162704, Citric Acid Anhydrous, Sipernat 50S, Vegetable Juice Color 4507 (BEET), Lo Han Guo Ext. (40% Magrosides).

Pill Form: Each material is bought in bulk. Then exact measurements are taken for each material using a measuring spoon and put into a capsule to make it a pill.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

What is claimed is:

1. A composition taken before, during, or after exercise; comprising: potassium; sodium; magnesium; chromium; vitamin B12; vitamin C; vitamin E; and sucrose.

2. The composition of claim 1 including between 200 and 500 mg of potassium; between 100 and 350 mg of sodium; between 60 and 200 mg of magnesium; between 1 and 20 mcg of chromium; between 35 and 200 mcg of vitamin B12; between 300 and 700 mg of vitamin C; between 3 and 25 mg of vitamin E; and between 2 and 20 mg of sucrose.

3. The composition of claim 1 including between 325 and 425 mg of potassium; between 175 and 225 mg of sodium; between 75 and 125 mg of magnesium; between 5 and 15 mcg of chromium; between 475 and 525 mcg of vitamin B12; between 475 and 525 mg of vitamin C; between 8 and 15 mg of vitamin E; and between 8 and 12 mg of sucrose.

4. The composition of claim 1 including approximately 400 mg of potassium; approximately 200 mg of sodium; approximately 100 mg of magnesium; approximately 0.01 mg or 10 mcg of chromium; approximately 1 mg or 100 mcg of vitamin B12; approximately 500 mg of vitamin C; approximately mg of vitamin E; and approximately 10 mg of sucrose.

5. A method of preparation of an orally ingestible blend, comprising the steps of:
obtaining in substantially powder form the following constituents: potassium; sodium; magnesium; chromium; vitamin B12; vitamin C; vitamin E; and, sucrose;
combining said constituents into a mixture.

6. The method of claim 5 wherein the mixture contains between 200 and 500 mg of potassium; between 100 and 350 mg of sodium; between 60 and 200 mg of magnesium; between 1 and 20 mcg of chromium; between 35 and 200 mcg of vitamin B12; between 300 and 700 mg of vitamin C; between 3 and 25 mg of vitamin E; and between 2 and 20 mg of sucrose.

7. The method of claim 5 wherein the mixture contains between 325 and 425 mg of potassium; between 175 and 225 mg of sodium; between 75 and 125 mg of magnesium; between 5 and 15 mcg of chromium; between 475 and 525 mcg of vitamin B12; between 475 and 525 mg of vitamin C; between 8 and 15 mg of vitamin E; and between 8 and 12 mg of sucrose.

8. The method of claim 5 wherein the mixture contains approximately 400 mg of potassium; approximately 200 mg of sodium; approximately 80 mg of magnesium; approximately 0.01 mg or 10 mcg of chromium; approximately 1 mg or 100 mcg of vitamin B12; approximately 500 mg of vitamin C; approximately mg of vitamin E; and approximately 10 mg of sucrose.

9. The method of claim 5 wherein the composition is encapsulated for oral ingestion.
